(19) United States
(12) Patent Application Publication
(10) Pub. No.: US 2019/0264229 A1
(43) Pub. Date: Aug. 29, 2019

(54) DEVICE, SYSTEM AND METHOD FOR INTRACELLULAR DELIVERY OF SUBSTANCES

(71) Applicant: Agency for Science, Technology and Research, Singapore (SG)

(72) Inventors: Yiqi SEOW, Singapore (SG); Weijie Cyrus BEH, Singapore (SG)

(73) Assignee: Agency for Science, Technology and Research, Singapore (SG)

(21) Appl. No.: 16/343,503

(22) PCT Filed: Oct. 20, 2017
(86) PCT No.: PCT/SG2017/050527

§ 371 Date: Apr. 19, 2019

(30) Foreign Application Priority Data
Oct. 21, 2016 (SG) 10201608846Y

(51) Int. Cl. C12N 15/87

(52) U.S. Cl. CPC C12N 15/87

(57) ABSTRACT

There is provided an attachment configured to detachably couple to a fluid extruding device, the attachment comprising: a membrane having a plurality of pores configured to allow a cell to pass through while inducing a mechanical stress to the cell such that the introduction of one or more substances into the cell is facilitated; and an engaging member for coupling to the outlet of the fluid extruding device such that the membrane is in fluid communication with the outlet of the fluid extruding device, wherein the fluid extruding device is configured to extrude fluid via mechanical actuation.

### TECHNICAL FIELD

The present disclosure relates broadly to a device, system and method for intracellular delivery.

### BACKGROUND

Cells used in research and commercial production of biologics are frequently engineered to give them new functions that could be useful for understanding basic biology or to produce molecules of therapeutic or commercial interest. One of the most crucial process that is required to engineer anything within a cell is the ability to get macromolecules, such as DNA (deoxyribonucleic acid) plasmids and proteins, into a cell.

Cell delivery technology has developed in the past decades into a huge industry, and the market for just DNA/RNA (ribonucleic acid) delivery for research alone is predicted to be worth over USD 900 million by 2020. Technology utilising amphiphilic polymers/molecules and electroporation has dominated the cell delivery industry, particularly for animal, insect and plant cells. Such approaches have been effective with a large number of cell types and there are a range of products that are slightly modified to be optimised for one cell type or the other. There are also other approaches that employ viral vectors for the delivery of nucleic acids.

As with any technology, there are limitations to these approaches. Amphiphilic polymers/molecules are toxic at high doses, can be expensive, and frequently require refrigerated storage. For certain sensitive cell types, toxicity can also be observed at much lower doses. Electroporation requires a buffer with very low ionic strength and is thus not compatible with cell culture. The single-use cartridge is also very expensive and inherently low-throughput. Moreover, the electroporator is expensive to purchase. Viruses are very efficient but can only deliver nucleic acids, and they are tedious and expensive to produce. The efficacy of each delivery system is further limited by the cells.

Methods using microfluidic devices have been developed to load nucleic acids, proteins, dyes and small molecules into cells through cell deformation and shear stress. However, microfluidic device manufacture is typically a very time-consuming and expensive process. Operation of such a system also requires dedicated instrumentation. It is certainly not clear how such a system can be miniaturised and simplified to be integrated with commonly used laboratory equipment and workflow.

Membranes with pores of similar size or smaller than the diameter of cells can theoretically be used to load nucleic acids, proteins, dyes and small molecules into cells through cell deformation and shear stress. However, chemically crosslinked filters used commonly for cell culture applications have tortuous paths which trap particles above the desired pore size and are unsuitable for inducing mechanical shear. Since there is a large distribution of pore sizes in each filter, cells also preferentially exit through the largest pore, limiting the degree of mechanical shear induced.

Thus, there is a need for a device, system and method for intracellular delivery which seek to address one or more of the above problems.

### SUMMARY

According to one aspect, there is provided an attachment configured to detachably couple to a fluid extruding device, the attachment comprising: a membrane having a plurality of pores configured to allow a cell to pass through while inducing a mechanical stress to the cell such that the introduction of one or more substances into the cell is facilitated; and an engaging member for coupling to the outlet of the fluid extruding device such that the membrane is in fluid communication with the outlet of the fluid extruding device, wherein the fluid extruding device is configured to extrude fluid via mechanical actuation.

The engaging member for coupling to the outlet of the fluid extruding device may form a substantially air tight seal when coupled to the outlet of the fluid extruding device.

The engaging member may comprise a pliant material for engaging a circumference of the outlet of the fluid extruding device.

The engaging member may be configured to couple to the outlet of the fluid extruding device via a luer lock fitting.

The fluid extruding device may be selected from the group consisting of a pipette, manually-operated micropipette, electronically-operated micropipette, burette, syringe, automated/semi-automated liquid handling system and liquid handling robotics.

The plurality of pores may define substantially linear paths through the membrane.

The plurality of pores may have pore dimensions that are substantially uniform across said plurality of pores.

The pore dimensions may be at least one of: average diameter, average cross sectional area and average path length through the membrane.

The plurality of pores may have an average pore diameter which is from 40% to 70% of the average diameter of the cell.

The attachment may further comprise: a filter having pores with an average diameter larger than those of the membrane for reducing the incidence of cell clumps or cell aggregates reaching the membrane.

The attachment may further comprise a retaining ring to hold the membrane in a substantially fixed position.

The attachment may further comprise one or more valves disposed adjacent to the membrane to facilitate movement of the cell and the one or more substances through the plurality of pores in a single direction.

According to another aspect, there is provided a method for introducing one or more substances into a cell, the method comprising: detachably coupling the outlet of a fluid extruding device to an engaging member of an attachment comprising a membrane having a plurality of pores, such that the fluid extruding device is in fluid communication with the membrane; passing the one or more substances and the cell through the plurality of pores to facilitate introduction of the one or more substances into the cell by inducing a mechanical stress to the cell, wherein the fluid extruding device is configured to extrude fluid via mechanical actuation.

The method may further comprise forming a substantially air tight seal when the engaging member is coupled to the outlet of the fluid extruding device.

The engaging member may comprise a pliant material for engaging a circumference of the outlet of the fluid extruding device.

The step of detachably coupling the outlet of a fluid extruding device to an engaging member of an attachment may comprise coupling via a luer lock fitting.

The fluid extruding device may be selected from the group consisting of a pipette, manually-operated micropipette, electronically-operated micropipette, burette, syringe, automated/semi-automated liquid handling system and liquid handling robotics.

The plurality of pores may define substantially linear paths through the membrane.

The plurality of pores may have pore dimensions that are substantially uniform across said plurality of pores.

The pore dimensions may be at least one of: average diameter, average cross sectional area and average path length through the membrane.

The plurality of pores may have an average pore diameter which is from 40% to 70% of the average diameter of the cell.

The method may further comprise a step of reducing the volume on one side of the membrane to create a pressure differential across the membrane to facilitate flow of the cell and the one or more substance through the plurality of pores.

The step of reducing the volume may be a manual step.

The method may further comprise passing the one or more substances and the cell through a filter having pores with an average diameter larger than those of the membrane for reducing the incidence of cell clumps or cell aggregates reaching the membrane.

According to another aspect, there is provided a fluid extruding system for introducing one or more substances into a cell, the system comprising: an attachment as disclosed herein; and a fluid extruding device detachably coupled to the attachment, wherein the fluid extruding device is configured to extrude fluid via mechanical actuation.

According to another aspect, there is provided a fluid extruding device for introducing one or more substances into a cell, the device comprising: a fluid flow chamber; a membrane disposed within the fluid flow chamber, the membrane having a plurality of pores configured to allow a cell to pass through while inducing a mechanical stress to the cell such that the introduction of one or more substances into the cell is facilitated; and an actuator for applying a reducing volume in the fluid flow chamber to create a pressure differential across the membrane to facilitate flow of fluid across the membrane.

### Definitions

The term “viable” as used herein refers to the ability of cells in culture to replicate under suitable culture conditions. The term as used herein also refers to cells which are alive in the culture at a particular time.

The term “non-viable” as used herein refers to cells that are not capable of replicating under any known conditions.

The term “substrate” as used herein is to be interpreted broadly to refer to any supporting structure.

The term “micro” as used herein is to be interpreted broadly to include dimensions from about 1 micron to about 1000 microns. Exemplary sub-ranges that fall within the term include but are not limited to the ranges of from about 10 micron to about 900 microns, from about 20 micron to about 800 microns, from about 30 micron to about 700 microns, from about 40 micron to about 600 microns, from about 50 micron to about 500 microns, from about 60 micron to about 400 microns, from about 70 micron to about 300 microns, from about 80 micron to about 200 microns, or from about 90 micron to about 100 microns.

The term “nano” as used herein is to be interpreted broadly to include dimensions less than about 1000 nm. Exemplary sub-ranges that fall within the term include but are not limited to the ranges of less than about 900 nm, less than about 800 nm, less than about 700 nm, less than about 600 nm, less than about 500 nm, less than about 400 nm, less than about 300 nm, less than about 200 nm, less than about 100 nm, less than about 50 nm, less than about 20 nm, or less than about 10 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “engaged”, “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

### DESCRIPTION OF EMBODIMENTS

Exemplary, non-limiting embodiments of a device, system and method for intracellular delivery are disclosed hereinafter.

There is provided a device in the form of an attachment for introducing one or more substances into a cell. The attachment may comprise one or more filters/membranes having specifically defined dimensions to deliver one or more substances into the cell as it passes through the membrane. The attachment may be configured to be detachably coupled to a fluid extruding device, e.g. syringes, pipettes and micropipettes which are used in the field of biotechnology. In some embodiments, the attachment may be directly coupled to the fluid extruding device. In some embodiments, the attachment may be an adapter.

Advantageously, the attachment may be capable of introducing one or more substances into a cell without the need for complex and/or bulky equipment/setup. The attachment may be designed to be manufactured using inexpensive material and for disposable single-use. This improves user-friendliness and allows the attachment to be easily integrated into existing laboratory workflow. While the attachment may not require the use of complex and/or bulky equipment/setup, it may also be amenable to automation via laboratory robotics employed in pharmaceutical and high throughput screens, allowing wider usage of the attachment. In various embodiments, it would be appreciated that the attachment is not a microfluidic device.

The one or more substances which can be introduced into a cell may include but is not limited to small molecules, nanoparticles, macromolecules, polynucleotides, oligonucleotides, plasmids, RNA, DNA, amino acids, peptides, proteins, polymers, drugs, growth factors, compositions of matter and combinations thereof. Depending on the nature of substance to be introduced, the one or more substance may be in nuclease-free medium, in protease-free medium, or in saline buffer.

Advantageously, the attachment as disclosed herein may allow substances that normally cannot enter a cell to be introduced into the cell, e.g. into the cytoplasm of the cell. It is recognised that chemical processing e.g. complexation with lipid or polymer based delivery systems or genetic processing in e.g. viral delivery systems may be required in order to stabilise and protect a substance during delivery and/or to allow the substance to penetrate the cell membrane when it typically cannot under normal physiological condition without modification. However, certain processing techniques may alter the properties of the substance to be delivered. In various embodiments, use of the attachment may advantageously obviate the need for undue processing of a substance prior to delivery.

The cell may include but is not limited to animal cell, plant cell, bacterial cell, protozoal cell, fungal cell, mammalian cell or human cell. In one embodiment, the cell is a mammalian cell. In a further embodiment, the cell is a human cell. The cell may remain in a viable form after passing through the membrane of the attachment as disclosed herein. Deformation of the cell may allow for uptake of substances with minimal cell death. Advantageously, cell death and loss during the delivery process using the attachment as disclosed herein may not be higher than comparable cell loading techniques known in the art. Cell viability can be measured using techniques such as MTT assay, trypan blue dye, flow cytometry etc. It would be appreciated that while the singular term for “cell” is used, the attachment as disclosed herein may allow introduction of one or more substances to a plurality of cells.

In various embodiments, the average cell density to be used with the attachment as disclosed herein may be at least about 10⁴ cells/ml, at least about 10⁵ cells/ml, or at least about 10⁶ cells/ml. In one exemplary embodiment, the average cell density to be used with the attachment is about 1×10⁶ cells/ml.

The attachment as disclosed herein may comprise a membrane having a plurality of pores configured to allow the cell to pass through while inducing a mechanical stress to the cell to facilitate the introduction of one or more substances into the cell. The mechanical stress may comprise shear stress and/or deformation of the cell and/or cellular membrane. The mechanical stress may produce a transient perturbation in the cell membrane of the cell passing therethrough, which may increase the permeability of the cell membrane to facilitate introduction of one or more substances. In various embodiments, the plurality of pores are configured to reliably induce a shear stress to the cell so as to facilitate the introduction of one or more substances into the cell. This is advantageous over chemically crosslinked filters that are unsuitable for inducing mechanical shear.

The plurality of pores may have pore dimensions that are substantially uniform across said plurality of pores. The pore dimensions may be at least one of: average diameter, average cross sectional area and average path length through the membrane. The pore dimensions may be in the micrometer range.

The plurality of pores may define substantially linear paths through the membrane. Each of the substantially linear paths may have a substantially uniform cross-sectional area throughout. It may be particularly advantageous to have pores that define substantially linear paths as compared to pores with tortuous paths which trap particles above the desired pore size and are unsuitable for inducing mechanical shear.

The substantially linear path may have a cross-sectional area with an average diameter that is capable of inducing mechanical stress on the cell, and yet not excessively small to rupture cells passing therethrough. In one embodiment, the average pore diameter may be approximately equal to the average diameter of the cell. In another embodiment, the average pore diameter may be less than the average diameter of the cell.

In various embodiments, the average diameter of the cross-section of the substantially linear path and/or the average pore diameter may be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, or at least about 150% of the average diameter of the cell.

In various embodiments, the average diameter of the cross-section of the substantially linear path and/or the average pore diameter may be from about 0.1 μm to about 5 μm, from about 0.5 μm to about 4 μm, from about 1 μm to about 3 μm, from about 2 μm to about 3 μm, from about 5 μm to about 20 μm, from about 6 μm to about 19 μm, from about 7 μm to about 18 μm, from about 8 μm to about 17 μm, from about 9 μm to about 16 μm, from about 10 μm to about 15 μm, from about 11 μm to about 14 μm, or from about 12 μm to about 13 μm.

In various embodiments, the average path length of the substantially linear path defined by the plurality of pores may be from about 5 μm to about 100 μm, from about 10 μm to about 95 μm, from about 15 μm to about 90 μm, from about 20 μm to about 85 μm, from about 25 μm to about 80 μm, from about 30 μm to about 75 μm, from about 35 μm to about 70 μm, from about 40 μm to about 65 μm, from about 45 μm to about 60 μm, or from about 50 μm to about 55 μm.

In various embodiments, the ratio of the average path length to the average pore diameter may be about 250:1, about 200:1, about 150:1, about 100:1, about 50:1, about 25:1, about 10:1, about 9:1, about 8:1, about 7:1, about 6:1, about 5:1, about 4:1, about 3:1, about 2:1, or about 1:1.

In various embodiments, the average thickness of the membrane may be from about 5 μm to about 100 μm, from about 10 μm to about 95 μm, from about 15 μm to about 90 μm, from about 20 μm to about 85 μm, from about 25 μm to about 80 μm, from about 30 μm to about 75 μm, from about 35 μm to about 70 μm, from about 40 μm to about 65 μm, from about 45 μm to about 60 μm, or from about 50 μm to about 55 μm.

Advantageously, a membrane having a plurality of pores with substantially uniform dimensions e.g. well-defined pore diameter and substantially linear path length may ensure that the mechanical stress experienced by the cells is substantially uniform. In addition, such a configuration may ensure that fluid, e.g. a payload/mixture comprising cells and one or more substances to be introduced, flows evenly across the plurality of pores.

The plurality of pores may be disposed in an area of the membrane that is accessible by the cell. In various embodiments, the diameter of the area of the membrane that is accessible by the cell may be in the range of from about 1 mm to about 45 mm, from about 2 mm to about 40 mm, from about 2 mm to about 35 mm, from about 2 mm to about 30 mm, from about 2 mm to about 25 mm, from about 2 mm to about 20 mm, from about 2 mm to about 15 mm, from about 2 mm to about 10 mm, from about 2 mm to about 5 mm, or from about 2 mm to about 4 mm. In one exemplary embodiment, the diameter of the area of the membrane that is accessible by the cell is about 4 mm. The flow resistance may also be substantially uniform across the area of the membrane that is accessible by the cells.

In various embodiments, the membrane may be capable of withstanding a pressure that allows a flow rate of from about 0.1 ml/s to about 2 ml/s, from about 0.2 ml/s to about 1.9 ml/s, from about 0.3 ml/s to about 1.8 ml/s, from about 0.4 ml/s to about 1.7 ml/s, from about 0.5 ml/s to about 1.6 ml/s, from about 0.6 ml/s to about 1.5 ml/s, from about 0.7 ml/s to about 1.4 ml/s, from about 0.8 ml/s to about 1.3 ml/s, from about 0.9 ml/s to about 1.2 ml/s, or from about 1.0 ml/s to about 1.1 ml/s across the area of the membrane that is accessible by said cell. In one exemplary embodiment, the membrane may be capable of withstanding a pressure that allows a flow rate of 1 ml/s across the area of the membrane that is accessible by said cell. In another exemplary embodiment, the attachment is capable of passing fluid through at a rate of at least (20 mm×cross sectional area of membrane) per second.

In various embodiments, the membrane may be capable of withstanding a pressure that allows a flow through velocity of from about 1 cm/s to about 30 cm/s, from about 2 cm/s to about 28 cm/s, from about 4 cm/s to about 26 cm/s, from about 6 cm/s to about 24 cm/s, from about 8 cm/s to about 22 cm/s, from about 10 cm/s to about 20 cm/s, from about 12 cm/s to about 18 cm/s, or from about 14 cm/s to about 16 cm/s across the area of the membrane that is accessible by said cell. In one exemplary embodiment, the membrane is capable of withstanding a pressure that allows a flow through velocity of from about 1 cm/s to about 30 cm/s.

It is recognised that the pressure applied to cause the cell to pass through the plurality of pores is inversely proportional to the area of the membrane accessible by the cell. Assuming that the flow rate remains constant, a decrease in the area would cause an increase in the pressure exerted on the membrane.

It would be appreciated that in various embodiments the pressure, flow rate and flow through velocity are set at levels that would allow cells to undergo mechanical stress without rupturing the cell. In various embodiments, the flow rate through the membrane is from about 0.1 ml/s to about 1 ml/s. In one exemplary embodiment, at a membrane porosity of about 40%, the flow through velocity is from about 5 cm/s to about 50 cm/s. In another exemplary embodiment, at a membrane porosity of about 1%, the flow through velocity is from about 2 m/s to about 20 m/s. Accordingly, in certain embodiments when the membrane porosity is about 40%, the flow velocity is about 50 cm/s and the flow rate through the membrane is about 1 ml/s; and when the membrane porosity is about 20%, the flow velocity is about 20 cm/s and the flow rate through the membrane is about 1 ml/s As will be appreciated from the foregoing, it would be understood that when one of: the porosity of the membrane, flow velocity or flow rate is varied, the other two parameters may be calculated proportionally based on the values provided in the above embodiments.

The membrane having a plurality of pores may be a track etched membrane fabricated using track etching technology. Track etched membranes have a plurality of pores which are formed using a combination of bombardment with charged particles and chemical etching, resulting in substantially straight tracks of defined diameters.

The track etched membrane may be made from any known material that is capable of being track-etched. The track etched membrane may be made from polymers which include but are not limited to polyesters, polystyrenes, aromatic polyesters, polycarbonates, polyolefins, including polyethylene, polyethylene terephthalate, polypropylene, vinyl plastics such as polyvinyl difluoride (PVDF), and cellulose esters such as cellulose nitrate, cellulose butyrate and cellulose acetate. In one embodiment, the track etched membrane comprises polycarbonate.

The attachment as disclosed herein may further comprise a housing/casing for holding the membrane. The housing may be cylindrically shaped, having an inner circumference defining an internal chamber and an outer circumference defining an external shape of the housing. The housing may comprise a first opening and a second opening disposed at substantially opposite ends of the housing. The first opening may be an inlet for receiving fluid e.g. cells and one or more substances to be delivered and the second opening may be an outlet for allowing fluid to exit from the housing, and vice versa. The membrane may be disposed within the internal chamber of the housing anywhere between the first opening and the second opening. The membrane may extend across the cross section of the internal chamber, dividing the internal chamber into a first sub-chamber and a second sub-chamber, the first and second sub-chambers being in fluid communication with each other.

The housing may comprise one or more flanges disposed on the inner circumference of the housing for supporting and maintaining the membrane in a fixed position. The membrane may be further secured to the housing using sealing means to seal the periphery of the membrane such that fluid is directed to flow through the plurality of pores and not around the periphery of the membrane. Sealing means may comprise using polydimethylsiloxane (PDMS), nitrocellulose, epoxy, cyanoacrylates, silicone, and other adhesives or the like. The housing may be tapered at one end e.g. at the first opening to form a tip for extruding fluid and the other end e.g. the second opening may be configured to be engagable to an outlet of a fluid extruding device.

The housing may be made from polymers which include but are not limited to polytetrafluoroethylene, polysulfone, polyethersulfone, polypropylene, polyethylene, fluoropolymers, cellulose acetate, polystyrene, polystyrene/acrylonitrile copolymer, PVDF, and combination thereof. It would be appreciated that the housing may advantageously possess qualities such as low material cost, ease of fabrication, ease of mass production, sterilisable, low toxicity to cells etc.

The attachment as disclosed herein may further comprise a retaining ring disposed within the internal chamber of the housing. The retaining ring may be positioned adjacent to the membrane to maintain the membrane in a substantially fixed position and to prevent displacement of the membrane when in use. An aperture may be formed at the centre of the retaining ring to expose an area of the membrane and render said area of the membrane accessible for cells to pass through. The diameter of the aperture may be varied to define the area of the membrane that is accessible to cells.

The attachment as disclosed herein may further comprise an engaging member for coupling to an outlet of a fluid extruding device such that the membrane is in fluid communication with the outlet of the fluid extruding device. The attachment may be capable of being detachably coupled to the outlet of the fluid extruding device via the engaging member.

The engaging member for coupling to the outlet of the fluid extruding device may form a substantially air tight seal when coupled to the outlet of the fluid extruding device. The engaging member may comprise a sufficiently pliant material such as an elastomer or plastic e.g. polypropylene, polyethylene for coupling to the outlet of the fluid extruding device. The engaging member may be in the form of an elastomeric ring or other grip-providing configurations for engaging a circumference of the outlet of the fluid extruding device. The circumference may be an inner circumference or an outer circumference of the outlet of the fluid extruding device. The engaging member may be configured to connect/engage the outlet of the fluid extruding device via a friction fit, a screw fit, a luer-lock fit or the like.

The attachment as disclosed herein may further comprise a filter/pre-filter e.g. fine mesh having pores with diameters that are larger than those of the plurality of pores of the membrane for reducing the incidence of unwanted particles reaching the membrane. Unwanted particles may comprise cell clumps, cell aggregates and debris. Accordingly, the cells reaching the membrane may be substantially unaggregated.

The attachment as disclosed herein may further comprise one or more valves disposed within the housing. The one or more valves may be a unidirectional/one-way valve for allowing a substantially unidirectional flow of fluid. In one exemplary embodiment, one unidirectional valve may be disposed adjacent to the membrane in substantially the same plane as the membrane. The unidirectional valve may be configured to facilitate fluid flow in a direction from the outlet of the housing to the inlet of the housing. In this configuration, the unidirectional valve provides a path of lesser resistance for fluid to flow in the facilitated direction instead of flowing through the membrane. Such a configuration may allow fluid to pass through the membrane in one direction from the inlet to the outlet for one or more times.

In another exemplary embodiment, a first unidirectional valve may be disposed adjacent to the membrane and may be configured to facilitate flow in a first direction from the inlet of the housing to the outlet of the housing. A second unidirectional valve may be disposed adjacent to the membrane in substantially the same plane as the membrane. The second unidirectional valve may be configured to facilitate flow in a second direction from the outlet of the housing to the inlet of the housing. Such a configuration may also allow fluid to pass through the membrane in one direction, i.e. the first direction, for one or more times.

The attachment as disclosed herein may be coupled to a fluid extruding device which may include but is not limited to a pipette, manually-operated micropipette, electronically-operated micropipette, burette, syringe, automated/semi-automated fluid/liquid handling systems and liquid handling robotics designed to use micropipette tips. The fluid extruding device may comprise an outlet configured to engage in fluid communication with the inlet of the attachment as disclosed herein. The outlet of the fluid extruding device may be shaped with the appropriate dimensions to connect/engage the inlet of the attachment via a friction fit, a screw fit or a luer-lock fit.

The fluid extruding device may be configured to extrude fluid via mechanical actuation, for example relying on displacement and movement of mechanical components such as a plunger to actuate (such as providing a driving force) and create a pressure differential to extrude the fluid. This is opposed to a gas pressured/pressurised system that utilizes the introduction of gas (with or without valves) to expel fluid. The fluid extruding device may further comprise an actuator in the form of a piston or plunger which may be actuated manually by a human operator or an automated/semi-automated robotic mechanism e.g. robotic arm. The piston or plunger may be configured to undergo a dispensing movement, causing fluid to be extruded e.g. from the body of a syringe or from a pipette tip. When connected to the attachment as disclosed herein, the dispensing movement of the piston or plunger may cause the volume within the internal chamber of the attachment to reduce, thus leading to an increase in pressure within the internal chamber of the attachment on the side of the membrane facing the inlet. This creates a pressure differential across the membrane and forces fluid e.g. mixture containing cells and one or more substances to pass through the plurality of pores in the membrane. The fluid extruding device may be a handheld fluid extruding device which may be portable. In various embodiments, the pressure differential across the membrane is not created or regulated by a gas supply.

There is also provided a method of introducing one or more substances into a cell. The method may comprise passing the cell across an attachment as disclosed herein, the attachment comprising one or more membranes having specifically defined dimensions to deliver one or more substances into the cell as it passes through the membrane. The method may comprise inducing a mechanical stress to the cell as the cell is passing through the membrane such that the introduction of one or more substances into the cell is facilitated.

The method may comprise passing the cell and the one or more substances across the membrane once. That is, passing the cell and the one or more substances from the outlet of a fluid extruding device to the inlet of the attachment, followed by passing the cell and the one or more substance across the membrane, and followed by extruding the cell and the one or more substance from the outlet of the attachment.

The method may comprise a step of reducing the volume of fluid on the side of the membrane facing the inlet of the attachment, thereby creating a pressure differential across the membrane and forcing the fluid e.g. the cell and the one or more substance to flow across the membrane. When connected to the attachment as disclosed herein, the dispensing movement of the piston or plunger may cause the volume within the internal chamber of the attachment to reduce, thus leading to an increase in pressure within the internal chamber of the attachment on the side of the membrane facing the inlet. This creates a pressure differential across the membrane and forces fluid e.g. mixture containing cells and one or more substances to pass through the plurality of pores in the membrane. It would be appreciated that in various embodiments, the step of applying a pressure differential across the membrane does not require use of a gas supply to apply pressure and/or create a pressure differential.

The method may comprise passing the cell and the one or more substances across the membrane a plurality of times. In one embodiment, the step of passing the cell and the one or more substances across the membrane a plurality of times may comprise passing across the membrane in single direction a plurality of times. The step of passing the cell and one or more substance across the membrane may comprise passing through a unidirectional valve disposed adjacent to the membrane to facilitate flow in a single direction for a plurality of times. The method may comprise a step of reducing the volume of fluid on the side of the membrane facing the inlet of the attachment, thereby creating a first pressure differential across the membrane which forces the fluid e.g. the cell and the one or more substance to flow across the membrane in a first direction from the inlet to the outlet of the housing. This may be followed by a step of increasing the volume of space on the side of the membrane facing the inlet of the attachment, thereby creating a second opposite pressure differential which draws fluid back into the housing, flowing through the unidirectional valve instead of the membrane. The aforementioned steps may be repeated to pass the cell and one or more substances across the membrane in single direction for a plurality of times.

In another embodiment, the step of passing the cell and the one or more substances across the membrane a plurality of times may comprise passing across the membrane in a first direction; and passing across the membrane in a second direction which is substantially opposite to the first direction. The method may comprise a step of reducing the volume of fluid on the side of the membrane facing the inlet of the attachment, thereby creating a first pressure differential across the membrane which forces the fluid e.g. the cell and the one or more substance to flow across the membrane in a first direction from the inlet to the outlet of the housing. This may be followed by a step of increasing the volume of space on the side of the membrane facing the inlet of the attachment, thereby creating a second opposite pressure differential which draws fluid back into the housing, flowing through the membrane in a second opposite direction. The aforementioned steps may be repeated to pass the cell and one or more substances across the membrane in both directions for a plurality of times.

The step of passing the cell across the membrane may be performed at a substantially constant flow rate. The substantially constant flow rate may be a predetermined flow rate. In various embodiments, the predetermined flow rate is from about 0.1 ml/s to about 2 ml/s, or from about 0.2 ml/s to about 1.9 ml/s, or from about 0.3 ml/s to about 1.8 ml/s, or from about 0.4 ml/s to about 1.7 ml/s, or from about 0.5 ml/s to about 1.6 ml/s, or from about 0.6 ml/s to about 1.5 ml/s, or from about 0.7 ml/s to about 1.4 ml/s, or from about 0.8 ml/s to about 1.3 ml/s, or from about 0.9 ml/s to about 1.2 ml/s, or from about 1.0 ml/s to about 1.1 ml/s.

In various embodiments, the step of passing the cell across the membrane may be performed at a flow through velocity of from about 1 cm/s to about 30 cm/s, or from about 2 cm/s to about 28 cm/s, or from about 4 cm/s to about 26 cm/s, or from about 6 cm/s to about 24 cm/s, or from about 8 cm/s to about 22 cm/s, or from about 10 cm/s to about 20 cm/s, or from about 12 cm/s to about 18 cm/s, or from about 14 cm/s to about 16 cm/s.

The method may further comprise passing the cell across a filter having pores with diameters that are larger than those of the membrane before passing the cell across the membrane, to reduce the incidence of unwanted particles reaching the membrane.

The method may further comprise checking the cell to determine if the one of more substances has been introduced into the cell after the step of passing the cell across the membrane.

There is also provided a fluid extruding system for introducing one or more substances into a cell. The fluid extruding system may comprise a fluid flow chamber; a membrane as disclosed herein disposed within the fluid flow chamber; and an actuator for applying a reducing volume in the fluid flow chamber to create a pressure differential across the membrane. The actuator may be in the form of a plunger. The fluid extruding system may allow bidirectional flow of cells across the membrane. The fluid extruding system may further comprise a unidirectional valve disposed within the fluid flow chamber for allowing a substantially unidirectional flow of cells across the membrane. The membrane disclosed within the fluid flow chamber may be disposable and replaceable. In various embodiments, the system disclosed herein is devoid of an external gas supply and/or gas regulator.

There is also provided a system for introducing one or more substances into a cell. The system may comprise a fluid extruding device; and an attachment as disclosed herein coupled to an outlet of the fluid extruding device. In one embodiment, there is provided an apparatus for delivery of molecules into cells. The apparatus may comprise a syringe; and a membrane in fluid communication with the syringe, wherein the membrane comprises pores with substantially uniform dimensions.

In one embodiment, there is provided a device that uses two filters to deliver nucleic acid/protein/macromolecule into eukaryotic cells that is designed to mate to micropipette tips by elastomer grip and syringe via luer locks.

There is also provided a method for delivery of one or more molecules into cells. The method may comprise loading the one or more molecules and the cells in a syringe; and syringing the molecules and the cells through a membrane in fluid communication with the syringe, wherein the membrane comprises pores with substantially uniform dimensions.

### BRIEF DESCRIPTION OF FIGURES

FIG. 1 is a schematic drawing of an attachment for introducing one or more substances into a cell in an exemplary embodiment.

FIG. 2 is a schematic drawing of an attachment attached to a pipette tip for introducing one or more substances into a cell in an exemplary embodiment.

FIG. 3 is a schematic drawing of a pipette tip for introducing one or more substances into a cell in an exemplary embodiment.

FIG. 4 is a schematic drawing of a pipette tip attachment for introducing one or more substances into a cell in an exemplary embodiment.

FIG. 5 is a schematic drawing of a pipette tip for introducing one or more substances into a cell in another exemplary embodiment.

FIG. 6 is a schematic flowchart of a method for introducing one or more substances into a cell in an exemplary embodiment.

FIG. 7 is a schematic drawing showing a method of introducing one or more substances into cells in another exemplary embodiment.

FIG. 8 is a schematic drawing showing a method of introducing one or more substances into cells in yet another exemplary embodiment.

FIG. 9A is a micrograph of HEK293T cells transfected with peGFP-C1 plasmid (scale bar=500 μm).

FIG. 9B is a micrograph of HEK293T cells transfected with peGFP-C1 plasmid (scale bar=50 μm).

FIG. 10 is a graph comparing the transfection efficiency of a setup with or without prefiltration.

FIG. 11 is a graph comparing the transfection efficiency for different loading concentrations of cells.

FIG. 12 is a graph comparing the transfection efficiency for different loading concentrations of plasmid.

FIG. 13 is a graph comparing the transfection efficiency for different filter pore sizes.

FIG. 14 is a graph comparing the transfection efficiency for different aperture/diameter of the filter area.

FIG. 15 is a graph comparing the transfection efficiency for different flow rates.

FIG. 16 is a graph comparing the transfection efficiency for different cell recovery times.

FIG. 17 is a micrograph of HEK293T cells transfected with peGFP-C1 and pDsRed-N1 plasmids (scale bar=100 μm).

FIG. 18 is a set of micrographs showing cells which have been syringed and cells which have been incubated (scale bar=100 μm).

FIG. 19A is a micrograph of H1 cells transfected with peGFP-C1 (scale bar=100 μm).

FIG. 19B is a micrograph showing another view of H1 cells transfected with peGFP-C1 (scale bar=100 μm).

FIG. 20 is a set of micrographs showing cells which have been syringed through filters with 3 or 5 μm pores.

FIG. 21 is a chart comparing the transfection efficiency of different methods of delivery of plasmid into cells.

FIG. 22 is a chart comparing the transfection efficiency of different substances into cells.

FIG. 23 is a chart comparing the transfection efficiency of delivering plasmids with and without a filter membrane in CHO cells.

### DETAILED DESCRIPTION OF FIGURES

Exemplary embodiments of the disclosure will be better understood and readily apparent to one of ordinary skill in the art from the following discussions and if applicable, in conjunction with the figures. It should appreciate that other modifications related to structural, material and mechanical changes may be made without deviating from the scope of the invention. Exemplary embodiments are not necessarily mutually exclusive as some may be combined with one or more embodiments to form new exemplary embodiments.

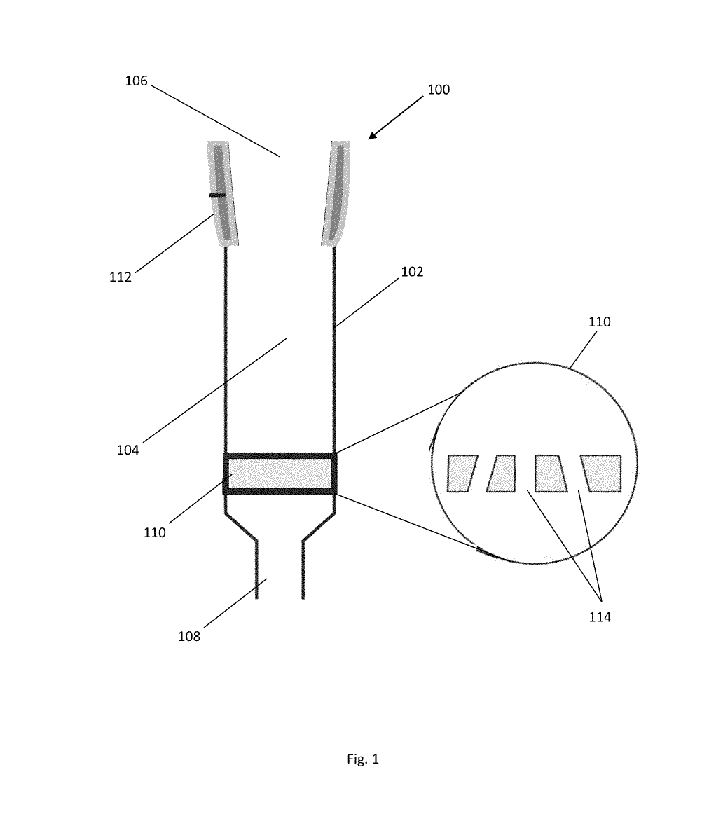

Referring to FIG. 1, there is shown a schematic drawing of an attachment 100 for introducing one or more substances into a cell in an exemplary embodiment. The attachment 100 comprises a housing 102 having an internal chamber 104 formed therein, an inlet 106 and an outlet 108 disposed at substantially opposite ends of the housing 102, a membrane 110 disposed within and extending across the transverse section of the internal chamber 104, and an engaging member 112 coupled to the inlet 104. The internal chamber 104, inlet 106, and outlet 108 are in fluid communication.

The membrane 110 comprises a plurality of pores 114 (see exploded view of membrane 110). Each of the plurality of pores 114 defines a substantially linear path and has a substantially uniform cross sectional area through the membrane 110. Each of the plurality of pores 114 has an average diameter that is predefined based on the average diameter of the cell passing through the membrane 110. Specifically, the average diameter of the plurality of pores 114 is about 3 to 20 μm and is defined such that it is approximately equal to or smaller than the average diameter of the cell. This induces a perturbation in the form of shear stress and/or cell deformation when the cell passes through the plurality of pores 114.

The membrane 110 is made of polycarbonate material and the plurality of pores 114 are made using track-etching technology

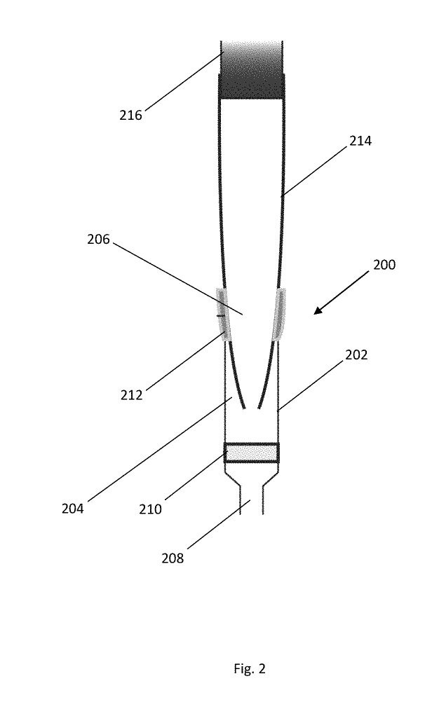

FIG. 2 is a schematic drawing of an attachment 200 attached to a pipette tip 214 for introducing one or more substances into a cell in an exemplary embodiment. The attachment 200 functions substantially similarly to the attachment 100 of FIG. 1. The attachment 200 comprises a housing 202 having an internal chamber 204 formed therein, an inlet 206 and an outlet 208 disposed at substantially opposite ends of the housing 202, a membrane 210 disposed within and extending across the transverse section of the internal chamber 204, and an engaging member 212 coupled to the inlet 204. The internal chamber 204, inlet 206, and outlet 208 are in fluid communication.

In the exemplary embodiment, the attachment 200 is detachably coupled to a pipette tip 214 which is in turn detachably coupled to a pipette 216. The cell and the one or more substances are delivered from the fluid extruding device into the attachment 200 via the inlet 206.

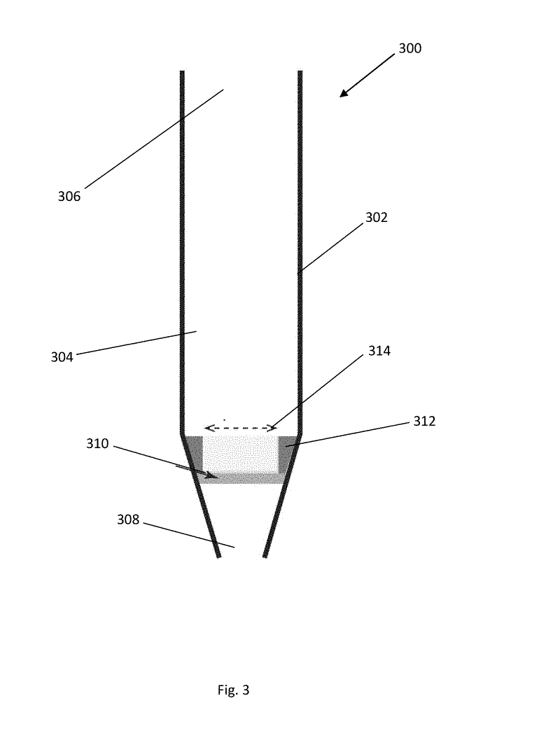

FIG. 3 is a schematic drawing of pipette tip 300 for introducing one or more substances into a cell in an exemplary embodiment. The pipette tip 300 comprises a housing 302 having an internal chamber 304 formed therein. The pipette tip 300 further comprises an inlet 306 configured to be detachably coupled to a pipette and an outlet 308 for extruding fluid e.g a mixture containing cells and one or more substances to be introduced. In the exemplary embodiment, a membrane 310 extending across the transverse section of the internal chamber 304 is disposed proximal to the outlet 308 and is held in a substantially fixed position by a retaining ring 312. The retaining ring 312 forms an aperture 314 through which an area of the membrane 316 is accessible by the cell.

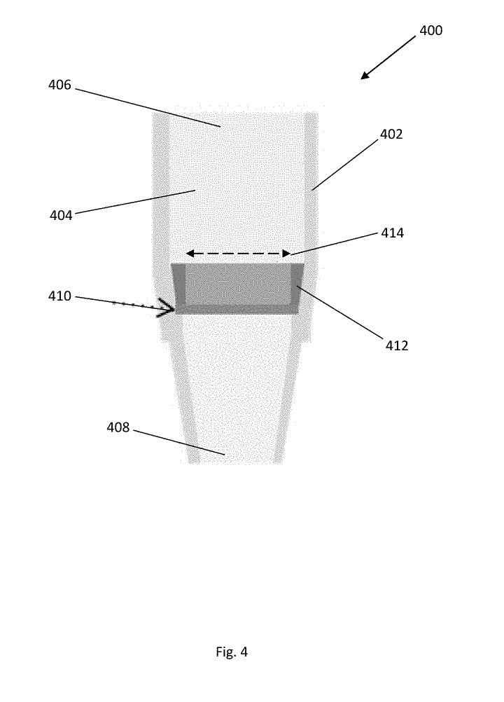

FIG. 4 is a schematic drawing of a pipette tip attachment 400 for introducing one or more substances into a cell in an exemplary embodiment. The pipette tip attachment 400 is configured as an adapter for detachably coupling to a pipette and comprises a housing 402 having an internal chamber 404 formed therein. The pipette tip attachment 400 further comprises an inlet 406 having a predefined diameter for fitting to the outlet of the pipette, and an outlet 408 for extruding fluid e.g a mixture containing cells and one or more substances to be introduced. In the exemplary embodiment, a membrane 410 extending across the transverse section of the internal chamber 404 is disposed proximal to the outlet 408 and is held in a substantially fixed position by a retaining ring 412. The retaining ring 412 forms an aperture 414 through which an area of the membrane 410 is accessible by the cell.

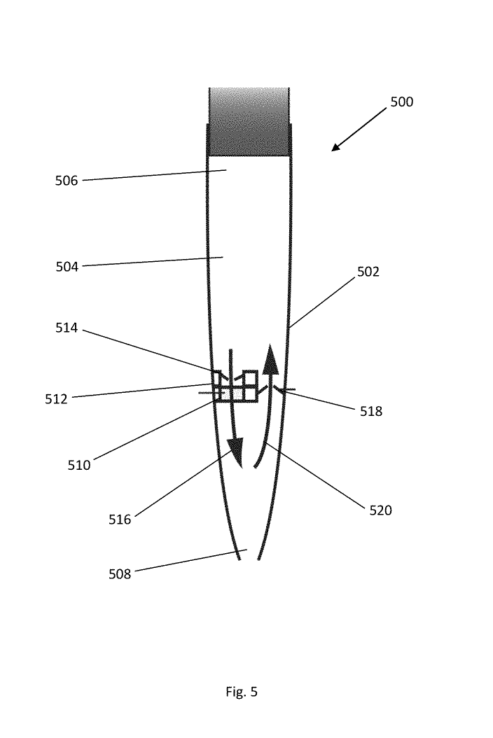

FIG. 5 is a schematic drawing of a pipette tip 500 for introducing one or more substances into a cell in another exemplary embodiment. The pipette tip 500 comprises a housing 502 having an internal chamber 504 formed therein. The pipette tip 500 further comprises an inlet 506 configured to be detachably coupled to a pipette and an outlet 508 for extruding fluid e.g a mixture containing cells and one or more substances to be introduced.

In the exemplary embodiment, a membrane 510 is disposed across a portion of the transverse section of the internal chamber 504 and is held in position by a retaining ring 512. A first one-way valve 514 is disposed adjacent to the membrane 510 nearer the inlet 506 and is configured to facilitate flow in a first direction 516 from the inlet 506 to the outlet 508. A second one-way valve 518 is disposed adjacent to the membrane 510 in substantially the same plane as the membrane 510. The second one-way valve 518 is configured to facilitate flow in a second direction 520 from the outlet 508 to the inlet 506. Such a configuration allows fluid to pass through the membrane 510 in one direction, i.e. the first direction 516.

When in use, a dispensing action on the pipette causes a reduction in the volume of fluid on the side of the membrane 510 facing the inlet 506, thereby creating a first pressure differential across the membrane 510 which forces fluid to flow across the membrane 510 in the first direction 516 from the inlet 506 to the outlet 508. A sucking action on the pipette causes an increase in the volume of space on the side of the membrane 510 facing the inlet 506, thereby creating a second opposite pressure differential across the membrane 510 which draws fluid back into the housing 502 via the outlet 508. Fluid that is drawn back into the housing 502 passes through the second one-way valve 518 in the second direction 520. The sucking action on the pipette causes the first one-way valve 514 to close and fluid cannot flow through the membrane 510 in the second direction 520. The dispensing and sucking action of the pipette may be repeated for a plurality of times to pass fluid containing the cell and one or more substances across the membrane 510 in a single direction for a plurality of times.

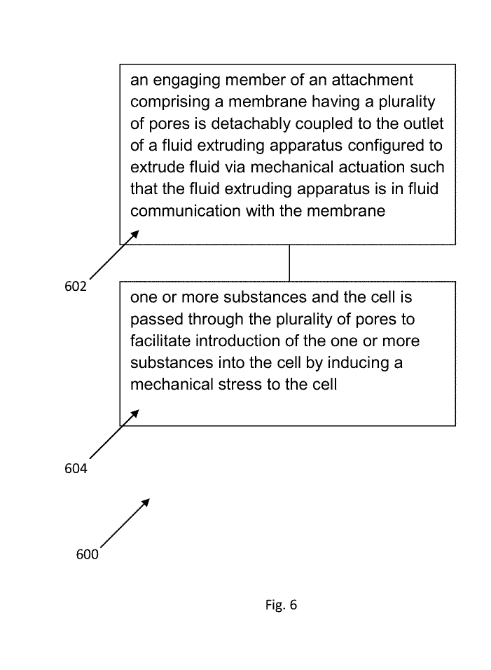

FIG. 6 is a schematic flowchart 600 of a method for introducing one or more substances into a cell in an exemplary embodiment. At step 602, an engaging member of an attachment comprising a membrane having a plurality of pores is detachably coupled to the outlet of a fluid extruding device configured to extrude fluid via mechanical actuation such that the fluid extruding device is in fluid communication with the membrane. At step 604, one or more substances and the cell is passed through the plurality of pores to facilitate introduction of the one or more substances into the cell by inducing a mechanical stress to the cell.

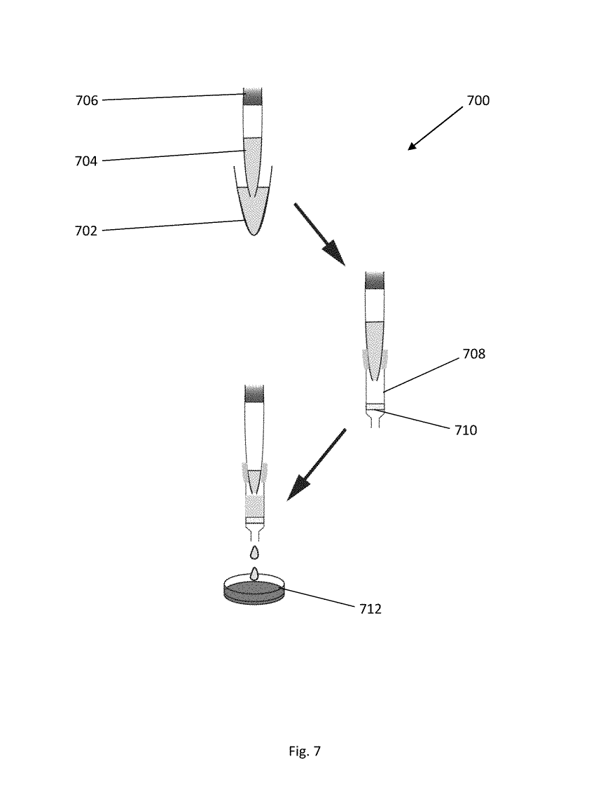

FIG. 7 is a schematic drawing showing a method 700 of introducing one or more substances into cells in another exemplary embodiment. A mixture 702 comprising cells and cargo, i.e. one or more substances to be introduced is drawn into a pipette tip 704 which is attached to a pipette 706. The pipette tip 704 containing the mixture 702 is coupled to an attachment 708. The attachment comprises a membrane 710 having a plurality of pores configured to allow cells to pass through while inducing a mechanical stress to the cells such that the cargo is delivered to the interior of the cells. The mixture 702 is collected in a collection dish 712 after passing through the membrane 710.

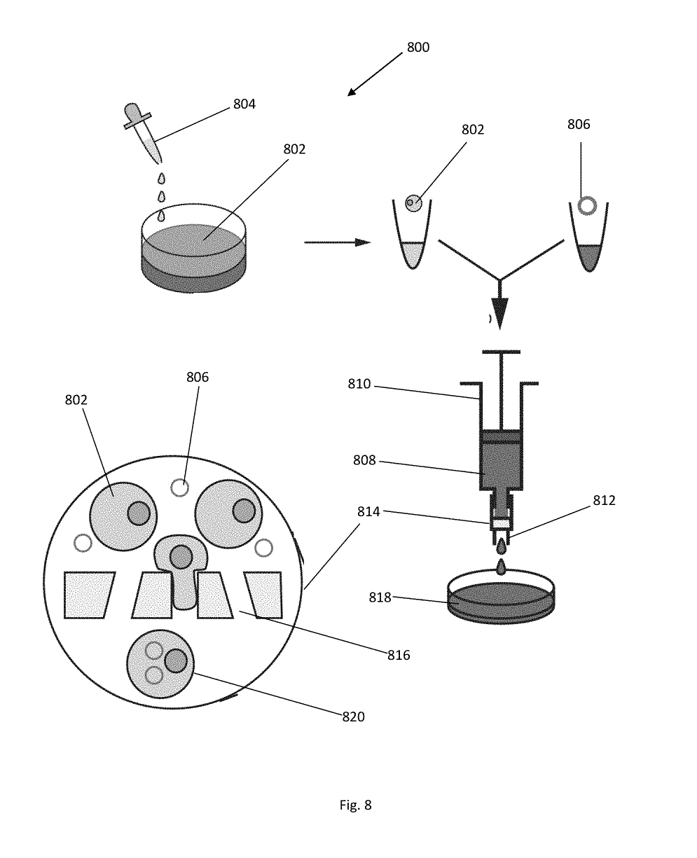

FIG. 8 is a schematic drawing showing a method 800 of introducing one or more substances into cells in yet another exemplary embodiment. Cells 802 in culture are first treated with a pretreatment mixture 804 comprising small molecules e.g. compositions for improving delivery efficiency. The cells 802 are incubated with the pretreatment mixture 804 for 6 hours followed by trypsinising to dislodge and harvest the cells 802 that are adhered to the culture dish. A solution containing cargo 806 to be delivered into the cells 802 is also prepared. The cells 802 are mixed together with the cargo solution 806 to form a mixture 808, followed by drawing the mixture 808 into a syringe 810. The syringe 810 containing the mixture 808 is detachably coupled to an attachment 812 comprising a membrane 814. The attachment 812 is directly coupled to the syringe 810.

Manual pressure is applied at a specific rate to pass the mixture through the membrane 814 without using gas pressure from a gas supply. The membrane 814 comprises a plurality of pores 816 with defined dimensions for applying mechanical stress on the cells 802 as they pass through the plurality of pores 816. As shown in the exploded view of the membrane 814, the cargo 806 is delivered to the interior of the cell 802 after passing through the plurality of pores 816 (see reference numeral 820).

### EXAMPLES

Exemplary embodiments of the disclosure will be better understood and readily apparent to one of ordinary skill in the art from the following examples, tables and if applicable, in conjunction with the figures.

In the following examples, a series of experiments was performed to evaluate the performance of the attachment as disclosed herein in introducing one or more substances into cells. Plasmids e.g. peGFP-C1 used in the experiments were procured from Clontech. Track-etched membranes having 25 mm in diameter were procured from Whatman (UK) and were housed in a 25 mm stainless steel holder (also from Whatman). The filter aperture was controlled by using polydimethylsiloxane (PDMS) to seal off the sides and a hole of the desired diameter was punched through the PDMS (Examples 1-6). HEK293T and CHO cells used in the study were grown in DMEM supplemented with 10% FCS and 1% Penicillin/Streptomycin at 37° C. in 5% CO₂.

### Example 1—eGFP Plasmid can be Delivered Via Filter Transfection

A transfection cartridge was assembled based on the setup as shown in FIG. 8. 12 μg of peGFP-C1, which expresses eGFP when transfected, was mixed with 1×10⁶ HEK293T cells in 1 ml of OptiMEM reduced serum media and syringed through a membrane with 10 μm pores through an aperture of 4 mm at 1 ml per second. The resultant cells were then left in OptiMEM for 5 minutes before 3 ml of cell culture medium. The cells were then imaged after 1 day.

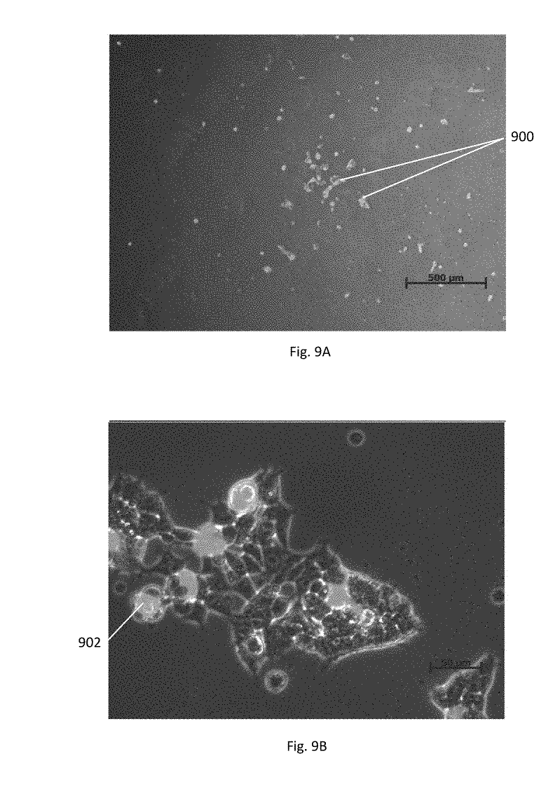

FIG. 9A is a micrograph of HEK293T cells transfected with peGFP-C1 plasmid (scale bar=500 μm). FIG. 9B is a micrograph of HEK293T cells transfected with peGFP-C1 plasmid (scale bar=50 μm). As seen in FIG. 9, a number of transfected cells (see reference numerals 900 and 902) were fluorescing green (see reference numerals 900 and 902), suggesting that the filter ‘transfection’ method works. It was observed that clogging occurred in the filter at the tail end of the syringing process because of cell clumps, which appeared with cells with very strong adherence to each other like HEK293T.

### Example 2—Defining Essential Parameters for Filter Transfection

To understand which parameter was important to getting high-efficiency delivery, each parameter was studied by modifying one parameter at a time in duplicates. By default, 5×10⁵ HEK293T cells were mixed with 5 μg of peGFP-C1 in 500 μl of OptiMEM and passed through a filter with 10 μm pores and 4 mm aperture at 1 ml per second. The resultant cells were then left in OptiMEM for 5 minutes before cell culture medium was added. The cells were then imaged 1 to 3 days after passing through the filter.

Cell numbers, pore size, aperture, speed of filtration, recovery time, plasmid concentration, and the need for prefiltration were compared to ascertain which parameter was important at influencing the efficiency of transfection. To measure the efficiency of transfection, the cells were imaged in both brightfield and green fluorescence in at least 3 separate fields and the total area covered by green fluorescence was then divided by the area covered by cells in each field, which gave an approximation as to the percentage of transfection. This measurement approach was used instead of counting cells without dissociation, which was difficult because the cells were frequently confluent and thus individual cells were hard to distinguish from each other.

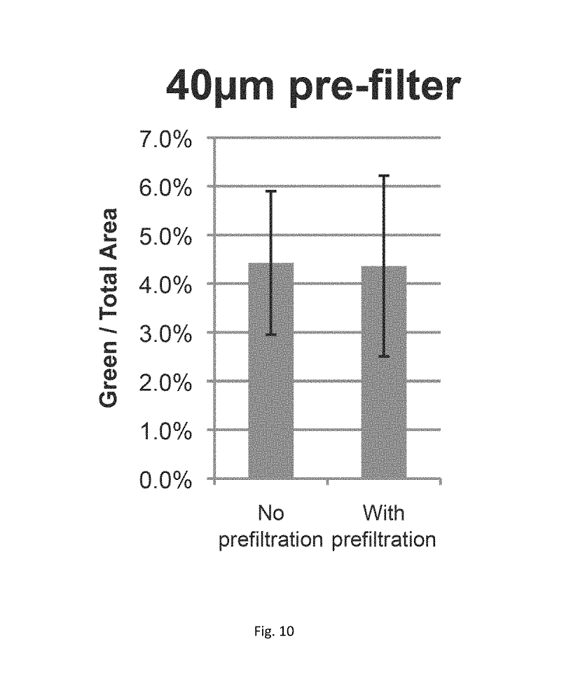

FIG. 10 is a graph comparing the transfection efficiency of a setup with or without prefiltration. As seen in FIG. 10, pre-filtration with a 40 μm filter has minimal effect to the transfection efficiency which was similar in the case with prefiltration and without prefiltration. However, it was observed that prefiltration has a significant effect on alleviating clogging of the filter.

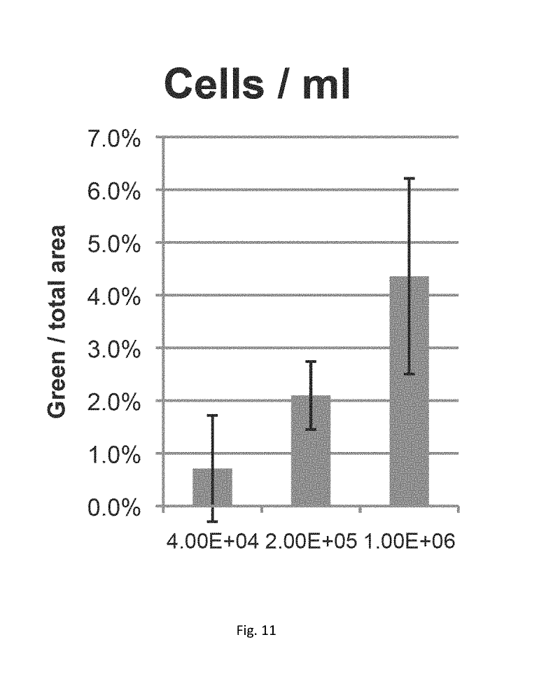

FIG. 11 is a graph comparing the transfection efficiency for different loading concentrations of cells. It was observed that lowering the concentration of cells also reduced the percentage of cells loaded, possibly due to the concentration of cells relative to cargo.

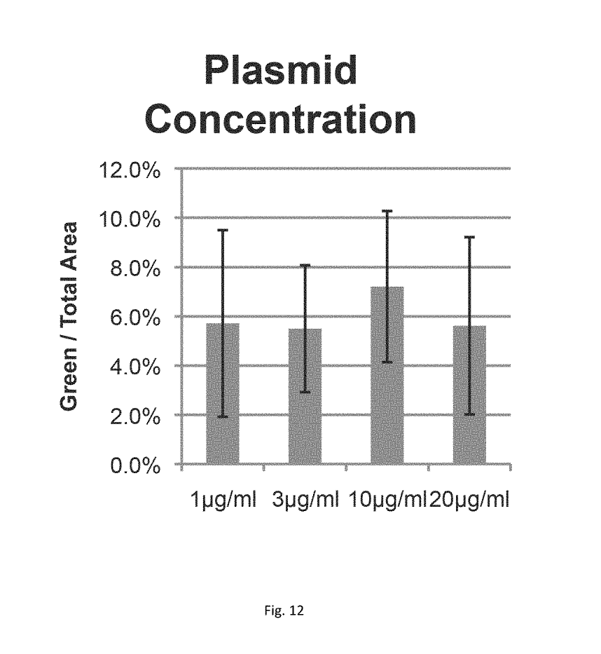

FIG. 12 is a graph comparing the transfection efficiency for different loading concentrations of plasmid. Surprisingly, it was observed that plasmid concentration has minimal effect on delivery efficiency. This suggests that plasmid concentration may not be limiting at the concentration range used although one would expect the delivery efficiency to drop as the concentration drops below 1 μg/ml.

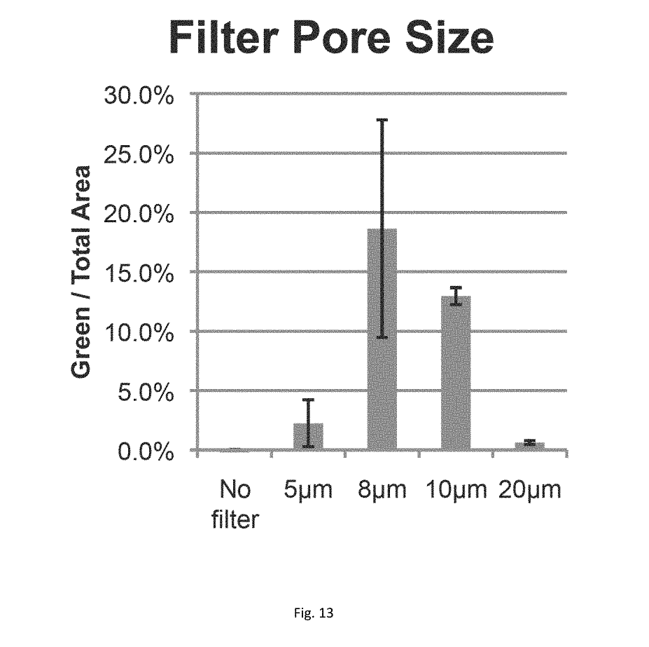

FIG. 13 is a graph comparing the transfection efficiency for different filter pore sizes. As HEK293T cells are about 14-20 μm in diameter, it is unlikely cells would survive if forced through a hole smaller than 5 μm in diameter. Therefore, pore sizes smaller than 5 μm was not used. As expected, without filtration, no fluorescence was seen. The results show that the optimal constriction was between 8 and 10 μm where the transfection efficiencies were significantly higher as compared to the other pore sizes. It was observed that few cells (less than 5% area with green fluorescence) survived and fluoresced after passing through 5 μm pores. There were also very few cells (less than 1% area with green fluorescence) fluorescing when passed through 20 μm meshes. However, it was surprisingly observed that 20 μm constriction was sufficient to get some plasmid into the cells, which suggests that shear stress rather than cell deformation alone causes the uptake of the plasmid.

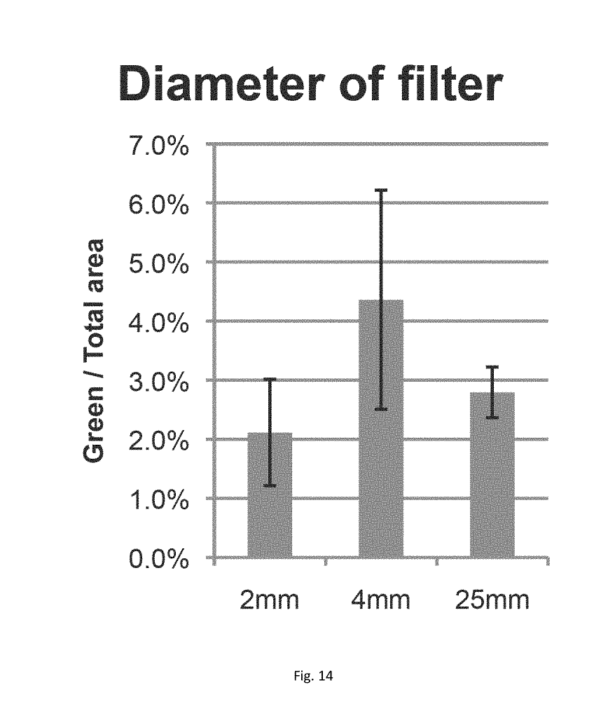

FIG. 14 is a graph comparing the transfection efficiency for different aperture/diameter of the filter area. Altering the aperture or diameter of the filter area should affect delivery efficiency as the filter surface area affects the pressure if flow rate is constant. As seen from FIG. 14, reduction of the filter area from 4 mm to 2 mm or an increase to 25 mm resulted in lower delivery efficiency. However, as delivery efficiency is still acceptable with a 2 mm aperture, this suggests that the system can be miniaturised to dimensions suitable for manufacturing into a syringe tip or a micropipette tip.

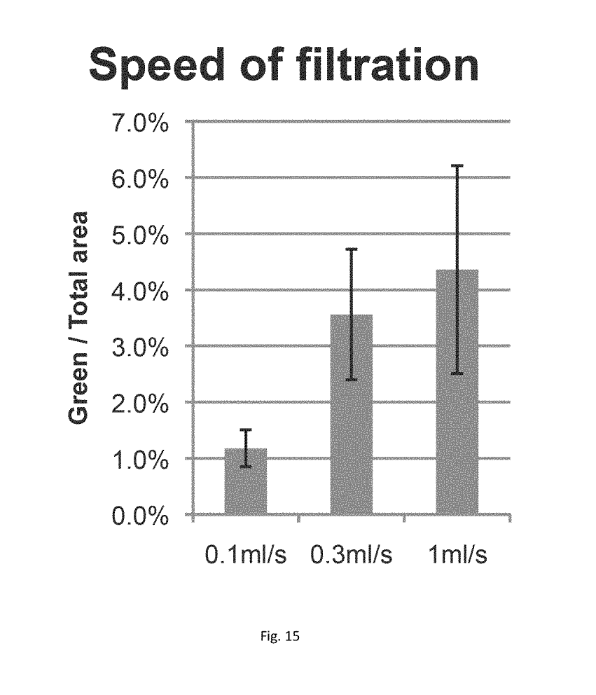

FIG. 15 is a graph comparing the transfection efficiency for different flow rates. Similar to alteration of aperture, slowing the flow rate down reduces the pressure and shear stresses. As seen from FIG. 15, reduction of flow rate to 0.3 ml/s and 0.1 ml/s reduced the delivery efficiency significantly. In combination, this suggests that the optimal flow rate is 1 ml/s for a membrane with 4 mm aperture.

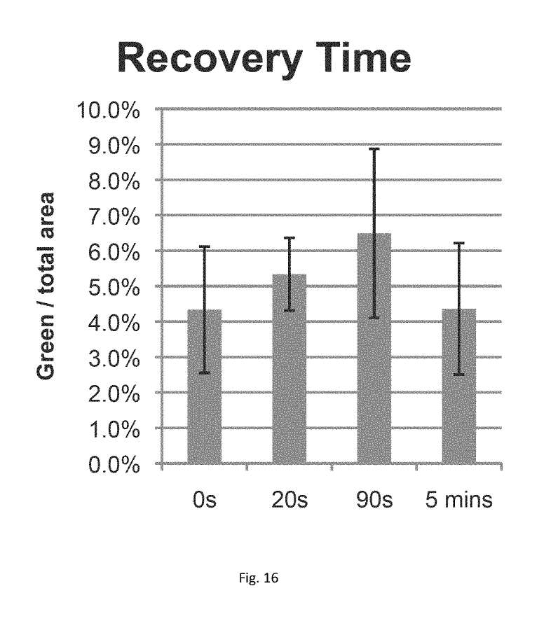

FIG. 16 is a graph comparing the transfection efficiency for different cell recovery times. It was observed that allowing time for the cells to recover after passing through the filter is apparently not necessary as the loading efficiency does not change significantly with different periods of recovery.

### Example 3—Multiple Plasmids can be Delivered Together

To investigate if two separate plasmids can be co-delivered into the same cells, 12 μg of pDsRed-N1, 12₄ of peGFP-C1 and 3×10⁵ HEK293T cells were syringed through 10 μm pores of 4 mm aperture at 1 ml/s in 1 ml of OptiMEM. The resultant cells were imaged 4 days after passing through the filter.

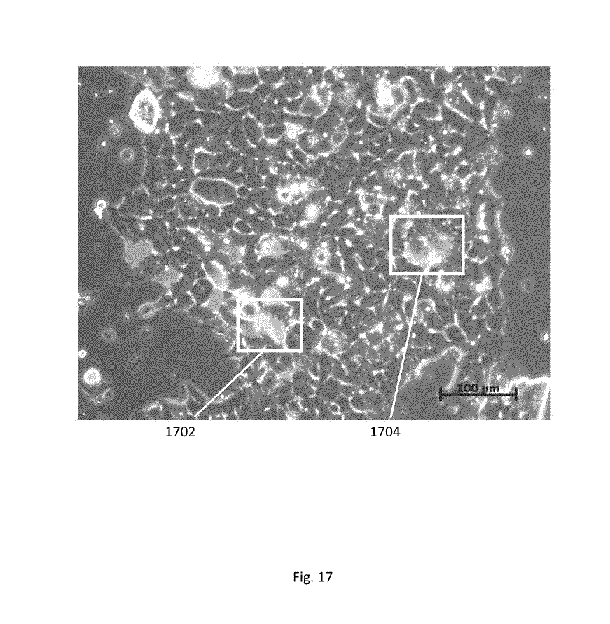

FIG. 17 is a micrograph of HEK293T cells transfected with peGFP-C1 and pDsRed-N1 plasmids (scale bar=100 μm). As seen from the micrograph, some cells were positive for both red and green fluorescent proteins (see highlighted areas 1702 and 1704). The results suggest that separate plasmids can be delivered together into the same cells. Interestingly, the number of cells positive for both DsRed and eGFP was higher than random independent distribution of eGFP-positive and DsRed-positive cells, suggesting that the transfection (number of positive cells) is limited by the number of cells made ‘permeable’ rather than the concentration of plasmid. Hence, improved designs of the cell shearing paths through innovative filter designs may increase the number of the cells made permeable by shear.

### Example 4—Filtration can Also Load Peptides into Cells

Experiments were performed to investigate if the method could also be used to load peptides into cells in a similar fashion. PM2 is a stapled peptide labeled with fluorescein and therefore it can be visualised under the microscope. 5 μM of PM2 was mixed with 1×10⁶ HEK293T cells in 1 ml of OptiMEM. One group of samples was incubated with the peptide only, the other group of samples was syringed through a 10 μm pore at 1 ml/s. The medium was changed out after 1 day and cells were washed thrice before imaging.

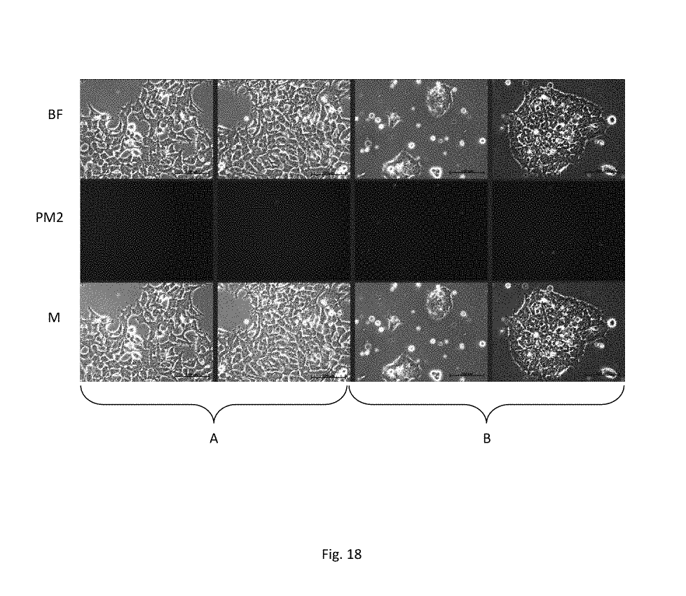

FIG. 18 is a set of micrographs showing cells which have been syringed and cells which have been incubated (scale bar=100 μm). Images at the top row were taken under bright field microscopy. Images at the middle row correspond to those at the top row and were taken under fluorescence microscopy. Images at the bottom row are processed images obtained by merging the images in the top and middle rows. The set of images under Group A belongs to the group of samples that was incubated with the peptide only. The set of images under Group B belong to the group of samples that was syringed through a 10 μm pore at 1 ml/s. As seen from the images in Group B, the presence of green fluorescence suggests that the peptide was taken up by the cells at much higher concentration after syringing through as compared to Group A samples which were merely incubated without syringing. However, some green fluorescence was still observed in the Group A samples, suggesting that there is some uptake with just incubation only.

### Example 5—Filtration can be Used to Load Difficult-to-Load Cell Types

Experiments were performed to investigate if the same method could work with other cell types, particularly difficult-to-transfect stem cells and primary cells. 1×10⁶ H1 cells was studied using a similar protocol as with the HEK293T cells.

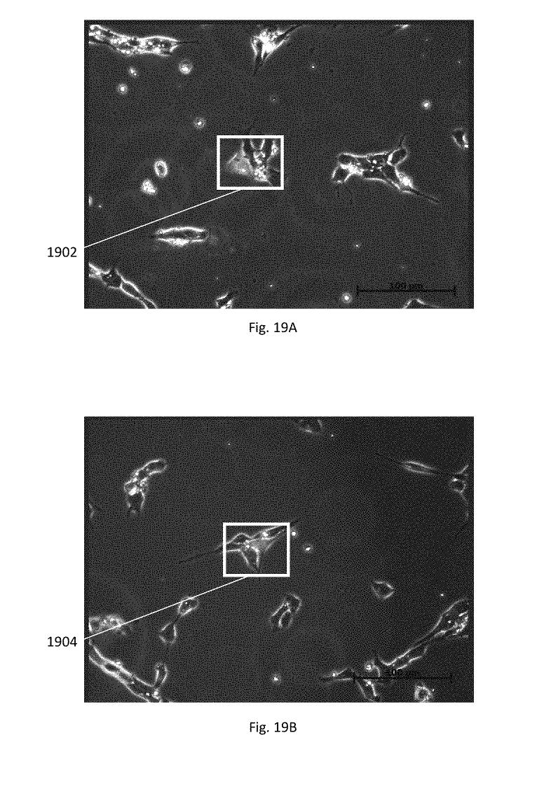

FIG. 19A is a micrograph of H1 cells transfected with peGFP-C1 (scale bar=100 μm). FIG. 19B is a micrograph showing another view of H1 cells transfected with peGFP-C1 (scale bar=100 μm). As seen from FIG. 19A and FIG. 19B, transfection of peGFP-C1 plasmids achieved effective delivery as well (see reference numerals 1902 and 1904).

Next, 10 μg peGFP-C1 with 2×10⁶ white blood cells (which are on average 6 μm in diameter) in 1 ml of medium and syringed through membranes of 3 or 5 μm pores with 4 mm aperture at 1 ml/s. One day later, the cells were imaged.

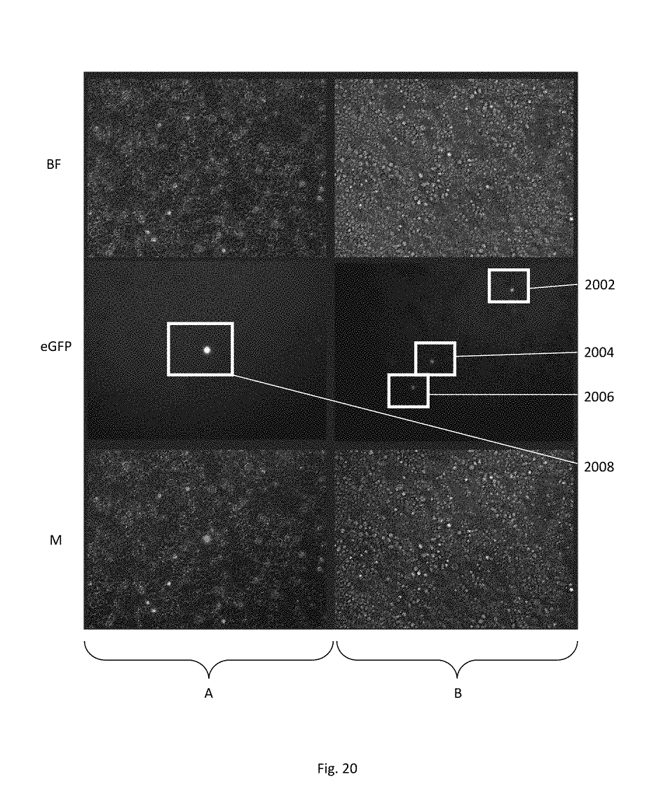

FIG. 20 is a set of micrographs showing cells which have been syringed through filters with 3 or 5 μm pores. Images at the top row were taken under bright field microscopy. Images at the middle row correspond to those at the top row and were taken under fluorescence microscopy. Images at the bottom row are processed images obtained by merging the images in the top and middle rows. The set of images under Group A belongs to the group of samples that was syringed through a filter with 3 μm pores. The set of images under Group B belong to the group of samples that was syringed through a filter with 5 μm pores. As seen from FIG. 20, a few cells were positive for eGFP fluorescence, suggesting that the method works for primary white blood cells (see reference numerals 2002, 2004, 2006 and 2008).

### Example 6—Filter can be Implemented in a Pipette Tip and Operated with Typical Pressures Used in a Manual/Automated Pipette Dispenser

In general, a syringe is not typically used in the laboratory setting for dispensing and transferring liquids. Experiments were thus performed to investigate if the filter would work with pipette tips instead. 1000 μl pipette tips were mated to syringe filters fitted with track-etched filters of 10 μm pore sizes in the manner as depicted in FIG. 2. 10 μg of peGFP-C1 was mixed with 0.4 million HEK293T in 500 μl of OptiMEM and the mixture was pipetted up and down through the filter using a 1 ml pipette in triplicates. This means that the cells will pass through the membrane twice during the entire process.

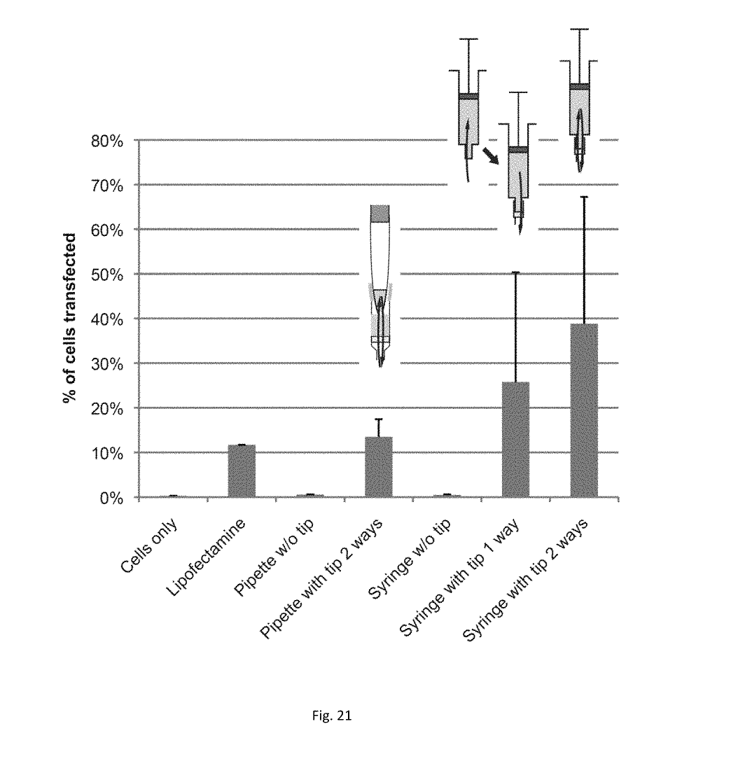

FIG. 21 is a chart comparing the transfection efficiency of different methods of delivery of plasmid into cells. Analysis of the percentage of cells with green fluorescence 2 days after treatment with flow cytometry showed that the pipette tip works as well as a lipofectamine 2000 control. Control samples that did not pass through the filter did not result in any cells exhibiting green fluorescence. When the same experiment was done with pipette tip, it seems that passing through the filter twice improved the efficiency of delivery although the effect was not statistically significant.

### Example 7—Pipette Tip Filters can be Used to Load a Variety of Cargo

To establish if other cargoes can be loaded with this method, 0.4 million HEK293T cells were mixed with 50 pmoles of fluorescein-labeled 21nt DNA oligonucleotide, 50 pmoles of fluorescein-labeled 21nt double-stranded siRNA, or 5 μg of fluorescein-dextran (MW 3000-5000) in 500 μl of OptiMEM. The mixture was aspirated and dispensed through the filter with the 1 ml pipette in triplicates. The mixture was then transferred into 2 ml of regular DMEM medium and the cells allowed to recover for 12 hours at 37° C. After which, the cells were washed 3 times with DMEM and collected for flow cytometry.

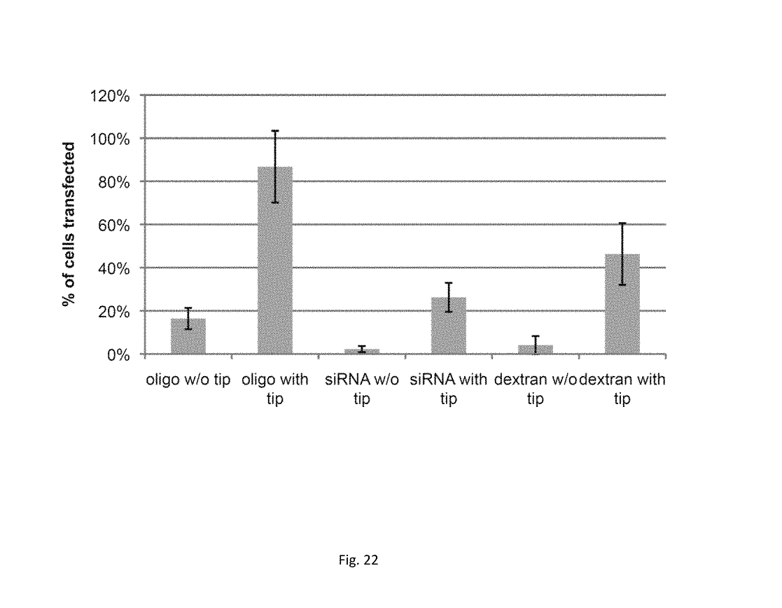

FIG. 22 is a chart comparing the transfection efficiency of different substances into cells. It is observed that the percentage of cells having a higher than background fluorescence after pipetting through the filter is clearly higher than the percentage of cells which fluoresced green when the cells do not pass through the filter for all types of cargo. This suggests that filtration can be used to deliver different types of cargo into HEK293T cells, including DNA, RNA and macromolecules (Dextran).

### Example 8—Other Cell Types can Also be Loaded with Plasmid Cargo Using Filtration

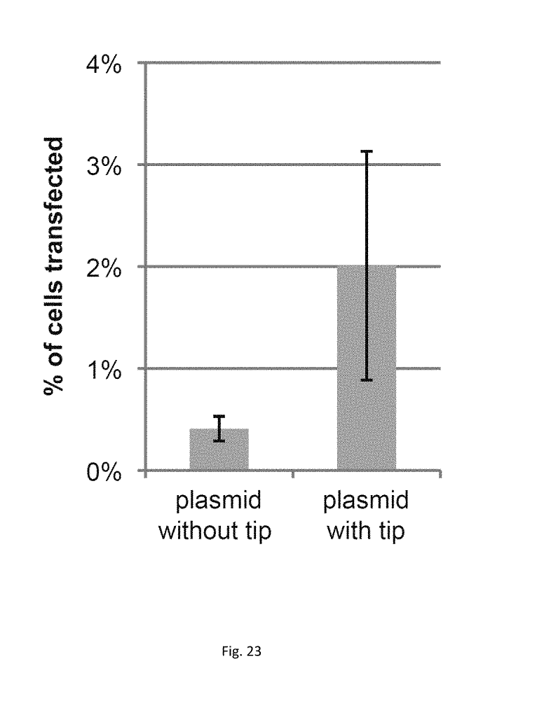

To establish if this method was broadly applicable to other mammalian cells, Chinese Hamster Ovary (CHO) cells were evaluated with peGFP-C1 plasmid. 0.2 million CHO cells were mixed with 10 μg of plasmid in 500 μl of OptiMEM and the mixture was aspirated and dispensed through a 10 μm filter membrane in the pipette setup shown in FIG. 2. FIG. 23 is a chart comparing the transfection efficiency of delivering plasmids with and without a filter membrane in CHO cells. It was observed that squeezing the CHO cells through the filter resulted in delivery of the plasmid into the cells as shown by the percentage of cells with fluorescence above the threshold.

### APPLICATIONS

Embodiments of the disclosure provided herein may provide a device in the form of an attachment configured to detachably couple to a fluid extruding device. In various embodiments, the attachment comprises a membrane having a plurality of pores configured to allow a cell to pass through while inducing a mechanical stress to the cell such that the introduction of one or more substances into the cell is facilitated.

Various embodiments of the present disclosure provide a cheaper and more effective option than electroporation.

Various embodiments of the present disclosure emphasise the use of disposable devices that does not require any additional instrumentation and/or manipulation, and can be plugged directly into existing cell culture workflow. Various embodiments of the set-up do not require gas supply such as a gas canister or bulky equipment in complicated setups that does not lend itself to scale-up. Various embodiments of the attachments disclosed herein are designed that can be attached to common syringes and micropipettes found in the lab and that are amenable to automation via laboratory robotics, e.g. liquid handling robotics and automated/semi-automated liquid handlers employed in pharmaceutical and high throughput screens, allowing wider usage of this technology.

For example, various embodiments of the attachment disclosed herein can be designed to be extremely user-friendly for research use as illustrated in an embodiment in FIG. 2. In this embodiment, the user only needs to attach the disposable ‘tip’ to a regular 1000 ml pipette, syringe up, syringe down and the cells will be transfected.

Even more advantageously, embodiments of the disclosure herein are capable of delivery into multiple cell types, as well as delivery of multiple agents. The delivery is also well tolerated and optimisation of delivery can be achieved. Substances such as gene, protein and small molecule may be delivered/transfected into cells by passing through a membrane having specifically defined pore dimensions to apply mechanical stress on the cells. In various embodiments, deformation of the cell allows for uptake with very little cell death. In various embodiments of the present disclosure, there may be some cell death and loss during the delivery process, but it is not higher than comparable cell loading techniques. Specific strategies that greatly improve the transfection efficiency are also described in the present disclosure.

In various embodiments of the present disclosure, instead of a microfluidic device, the inventors chose to subject cells to mechanical shear using a membrane. In order to ensure that the mechanical stress experienced by the cells is uniform, various embodiments of the disclosure provide membranes with pore size and path length that are well-defined with small variation. These requirements may be fulfilled by track etched membranes, which are made with bombardment of particles, resulting in straight tracks of defined diameter. In various embodiments, given the uniformity of the pore dimensions (length and diameter), the cell/payload mixture will flow evenly across the pores.

Exemplary embodiments of present disclosure provide different ways of subjecting cells to mechanical stress. One exemplary embodiment may be used as shown in FIG. 2 wherein a filter attachment comprising a membrane can be fitted to a pipette tip. In this embodiment, an elastomer seal is disposed on the filter attachment to provide a fluid-tight seal between the filter attachment and the pipette tip. When in use, the pipette tip first draws up a volume of solution containing cells with cargo/substance to be introduced. The pipette tip is then fitted with the filter attachment and pressure is applied via the pipette to eject the solution through the membrane in the filter attachment.

In yet another embodiment, the membrane for introducing substances into a cell can be incorporated into the flow chamber of a syringe/pipette, or attached to the tip of a syringe/pipette. As shown in FIG. 3 and FIG. 4, the membrane is disposed within the flow chamber of the syringe/pipette, held in place by a retaining ring. The aperture (i.e. portion of the membrane in fluid communication with the solution containing cells and substance to be introduced) can be set by, for example, adjusting the position of the membrane within the flow chamber.

It will be appreciated by a person skilled in the art that other variations and/or modifications may be made to the embodiments disclosed herein without departing from the spirit or scope of the disclosure as broadly described. The present embodiments are, therefore, to be considered in all respects to be illustrative and not restrictive.

1. An attachment configured to detachably couple to a fluid extruding device, the attachment comprising:
a membrane having a plurality of pores configured to allow a cell to pass through while inducing a mechanical stress to the cell such that the introduction of one or more substances into the cell is facilitated; and
an engaging member for coupling to the outlet of the fluid extruding device such that the membrane is in fluid communication with the outlet of the fluid extruding device,
wherein the fluid extruding device is configured to extrude fluid via mechanical actuation.

2. The attachment of claim 1, wherein the engaging member for coupling to the outlet of the fluid extruding device forms a substantially air tight seal when coupled to the outlet of the fluid extruding device, optionally wherein the engaging member comprises a pliant material for engaging a circumference of the outlet of the fluid extruding device, and optionally wherein the engaging member is configured to couple to the outlet of the fluid extruding device via a luer lock fitting.

3. (canceled)

4. (canceled)

5. The attachment of claim 1, wherein the fluid extruding device is selected from the group consisting of a pipette, manually-operated micropipette, electronically-operated micropipette, burette, syringe, automated/semi-automated liquid handling system and liquid handling robotics.

6. The attachment of claim 1, wherein the plurality of pores define substantially linear paths through the membrane.

7. The attachment of claim 1, wherein the plurality of pores have pore dimensions that are substantially uniform across said plurality of pores, optionally wherein the pore dimensions are at least one of: average diameter, average cross sectional area and average path length through the membrane.

8. (canceled)

9. The attachment of claim 1, wherein the plurality of pores has an average pore diameter which is from 40% to 70% of the average diameter of the cell.

10. The attachment of claim 1, further comprising:
a filter having pores with an average diameter larger than those of the membrane for reducing the incidence of cell clumps or cell aggregates reaching the membrane.

11. The attachment of claim 1, further comprising a retaining ring to hold the membrane in a substantially fixed position.

12. The attachment of claim 1, further comprising one or more valves disposed adjacent to the membrane to facilitate movement of the cell and the one or more substances through the plurality of pores in a single direction.

13. A method for introducing one or more substances into a cell, the method comprising:
detachably coupling the outlet of a fluid extruding device to an engaging member of an attachment comprising a membrane having a plurality of pores, such that the fluid extruding device is in fluid communication with the membrane; and
passing the one or more substances and the cell through the plurality of pores to facilitate introduction of the one or more substances into the cell by inducing a mechanical stress to the cell,
wherein the fluid extruding device is configured to extrude fluid via mechanical actuation.

14. The method of claim 13, further comprising forming a substantially air tight seal when the engaging member is coupled to the outlet of the fluid extruding device, optionally wherein the engaging member comprises a pliant material for engaging a circumference of the outlet of the fluid extruding device, and optionally wherein the step of detachably coupling the outlet of a fluid extruding device to an engaging member of an attachment comprises coupling via a luer lock fitting.

15. (canceled)

16. (canceled)

17. The method of claim 13, wherein the fluid extruding device is selected from the group consisting of a pipette, manually-operated micropipette, electronically-operated micropipette, burette, syringe, automated/semi-automated liquid handling system and liquid handling robotics.

18. The method of claim 13, wherein the plurality of pores define substantially linear paths through the membrane.

19. The method of claim 13, wherein the plurality of pores have pore dimensions that are substantially uniform across said plurality of pores, optionally wherein the pore dimensions are at least one of: average diameter, average cross sectional area and average path length through the membrane.

20. (canceled)

21. The method of claim 13, wherein the plurality of pores has an average pore diameter which is from 40% to 70% of the average diameter of the cell.

22. The method of claim 13, further comprising a step of reducing the volume on one side of the membrane to create a pressure differential across the membrane to facilitate flow of the cell and the one or more substance through the plurality of pores, optionally wherein the step of reducing the volume is a manual step.

23. (canceled)

24. The method of claim 13, further comprising passing the one or more substances and the cell through a filter having pores with an average diameter larger than those of the membrane for reducing the incidence of cell clumps or cell aggregates reaching the membrane.

25. (canceled)

26. A fluid extruding device for introducing one or more substances into a cell, the device comprising:
a fluid flow chamber;
a membrane disposed within the fluid flow chamber, the membrane having a plurality of pores configured to allow a cell to pass through while inducing a mechanical stress to the cell such that the introduction of one or more substances into the cell is facilitated;
and an actuator for applying a reducing volume in the fluid flow chamber to create a pressure differential across the membrane to facilitate flow of fluid across the membrane.
